(19) United States
(12) Patent Application Publication
(10) Pub. No.: US 2021/0275020 A1
(43) Pub. Date: Sep. 9, 2021

(54) NON-CONTACT AIR ESTHESIOMETER

(71) Applicant: Baylor College of Medicine, Houston, TX (US)

(72) Inventors: Stephen C. Pflugfelder, Houston, TX (US); Chia-Kai Chu, Houston, TX (US)

(73) Assignee: Baylor College of Medicine, Houston, TX (US)

(21) Appl. No.: 16/318,088

(22) PCT Filed: Jul. 18, 2017
(86) PCT No.: PCT/US17/42638

§ 371 Date: Jan. 15, 2019

Related U.S. Application Data
(60) Provisional application No. 62/363,720, filed on Jul. 18, 2016.

(51) Int. Cl. A61B 3/16; A61B 3/00

(52) U.S. Cl. CPC A61B 3/165; A61B 3/0083; A61B 3/0008

(57) ABSTRACT

Embodiments of the technology developed are a non-contact air esthesiometer used for measuring corneal sensitivity. In certain embodiments, the apparatus takes the OKI DX-255 Basic Digital Fluid Dispenser and modifies it for use to produce a 2-second stream of room-temperature air directed at the center of a patient's cornea. The input to the device is a compressed air tank, IN which can be easily changed, connected to an inline filter. The output is a hose line connected to a valve that permits finer adjustments in airflow rate to a disposable 200-microliter-filter pipette tip. This outlet tip is secured with self-setting rubber and housed in a metal stand with horizontal and vertical travel that can be directly mounted to a standard slit lamp. Four red LED lights were placed around the air outflow that can be used for patient fixation and alignment on the central cornea.

### CROSS-REFERENCE TO RELATED PATENT APPLICATIONS

This application claims the benefit of U.S. Provisional Application No. 62/363,720 to Pflugfelder et al. filed Jul. 18, 2016 and entitled “NON-CONTACT AIR ESTHESIOMETER” which is hereby incorporated by reference herein.

### FIELD OF THE DISCLOSURE

The instant disclosure relates to esthesiometers. More specifically, portions of this disclosure relate to air esthesiometers.

### BACKGROUND

Tear dysfunction is a prevalent disorder caused by decreased tear production, excessive evaporation or an altered distribution. Patients with tear dysfunction often experience irritation symptoms such as dryness, foreign body sensation, and burning however, paradoxically certain patients with moderate to severe ocular surface disease have a paucity of irritation symptoms. Patients with tear dysfunction may also complain of blurred and fluctuating vision, photophobia and frequent blinking. Increased frequency of blinking has been previously noted in patients with tear dysfunction; however, the factors contributing to the increased blink rate have not been established and may be influenced by the source of tear dysfunction. Studies evaluating tear dysfunction following LASIK have reported a decrease in blink rate. Although LASIK is known to cause corneal hyposensitivity which is often transient, no reduction in corneal sensitivity was found in one study, while hyperesthesia was measured in subjects with concurrent dry eye disease after LASIK.

The current industry standard method to measure corneal sensitivity is the Cochet-Bonnet esthesiometer, which relies on a fine nylon filament, the length of which can be varied to apply different intensities of stimulus, to come in contact with the corneal surface. Given that it only activates the mechanoreceptors on the surface of the eye, it underestimates corneal sensitivity and is unable to detect subtle changes in sensitivity, particularly at higher sensitivity levels. Furthermore, stimulus reproducibility is problematic due to practical difficulties in alignment, placement, and replication of the force applied to the nylon filament, in addition to the effects of ambient humidity and aging on the nylon itself. The tip is also difficult to sterilize.

### SUMMARY

A non-contact instrument would allow for superior stimulus reproducibility and better control over stimulus characteristics, in addition to the ability of activating all three types of neuro-receptors on the ocular surface. In one embodiment, a non-contact instrument for measuring sensitivity may be a non-contact air esthesiometer used for measuring corneal sensitivity. The apparatus may produce a 2-second stream of room-temperature air directed at the center of a patient's cornea. The input to the device may be a compressed air tank, which can be easily changed, connected through an inline filter to a hose line connected to a valve that permits finer adjustments in airflow rate to a disposable 200-microliter-filter pipette tip. This outlet tip may be secured with self-setting rubber and housed in a metal stand with horizontal and vertical travel that can be directly mounted to a standard slit lamp. In some embodiments, four red LED lights are placed around the air outflow that can be used for patient fixation and alignment on the central cornea.

A testing protocol for measuring corneal sensitivity using a non-contact instrument, such as in embodiments described herein, may include setting the compressed air tank and device to a set pressure output of 3 psi. The device is then programmed to have a cycle time of 2.000 seconds. The inline valve is then adjusted to 90 degrees counter-clockwise from the closed position. The slit lamp with the mounted air jet tip is then moved to the furthest position away from the patient's head and locked into place. The patient then places his/her chin in the chinrest and forehead against the strap of the slit lamp. The metal stand is then adjusted to the correct height to align with the center of the patient's cornea and then secured. The slit lamp is then moved left and right to line with the center of the patient's eye and then secured. There are 4 small red LEDs surrounding the tip that reflect off the cornea and can assist in centering the air jet on the center of the cornea. The patient is then instructed to close his eyes. The metal shaft is then advanced until the pipette tip comes in contact with the eyelid. Once contact has been confirmed, the metal shaft is retracted back 4 mm, as visible from the gradations on the side of the metal shaft. The patient is then instructed to open his eyes, look straight ahead, and try not to blink. The foot pedal is then depressed to release an air stream. The patient is asked if air jet was detected and if so, to describe the sensation produced from the air stream. If patient did not feel the air stream, the patient is asked to close his eyes while the needle valve is then adjusted counter-clockwise in increments of 45 degrees to increase the intensity of the stimulus, and the protocol repeated until the patient detects the air stream.

According to one embodiment, an apparatus (e.g., a non-contact air esthesiometer) may include a compressed air source and a line coupled to the compressed air source and configured to couple to an outlet tip, wherein the line is configured to supply compressed air from the compressed air+source through the line to exit through the outlet tip

In certain embodiments, the apparatus may also include a valve coupled between the line and the outlet tip and configured to adjust an airflow rate of the compressed air exiting through the outlet tip; the valve may be configured to provide an airflow rate of approximately 3 psi at the outlet tip; the apparatus may also include an inline filter coupled between the compressed air source and the line; the compressed air source may be one of a compressed air tank and an air compressor, or a combination thereof; the apparatus may also include a stand configured to secure the outlet tip and configured to provide horizontal and vertical movement of the outlet tip; the metal stand may also include a housing for the outlet tip that has four light emitting diode (LED)-based bulbs surrounding the outlet tip; and/or the outlet tip may be a disposable 200-microliter-filter pipette tip; the apparatus may also include a user input device (e.g., a foot pedal), wherein activation of the user input device triggers a 2-second stream of air to exit the outlet tip.

According to another embodiment, a method may include a method of operating an air esthesiometer including the steps of placing a patient's head into a stand or slit lamp chin rest, adjusting the stand or slit lamp horizontally and vertically to align an outlet tip with a center of the patient's eye, advancing the outlet tip towards the patient's eye to a desired distance from the patient's eye, triggering an outlet of compressed air through the outlet tip towards the patients' eye, and/or recording the patient's response to the compressed air.

In some embodiments, the method may further include increasing a pressure of the air from the outlet tip; and repeating the steps of triggering the outlet of compressed air and recording the patient's response to the compressed air; the step of adjusting the slit lamp to align the outlet tip with the center of the patient's eye comprises using the reflection of LED bulbs in the patient's cornea; and/or the step of triggering an outlet of compressed air comprises outputting two seconds of compressed air at approximately 3 psi towards the patient's eye.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 illustrates components of an air jet esthesiometer according to one embodiment of the disclosure that may be used to determine corneal sensitivity in patients with tear dysfunction in some embodiments of the disclosure.

FIG. 2 is a graph illustrating Correlation between corneal sensitivity measures with both one embodiment of an air jet esthesiometer and conventional Cochet-Bonnet esthesiometers in patients with tear dysfunction and in normal controls, where correlation between the air esthesiometer and Cochet-Bonnet was evaluated in all subject and a significant correlation was found to exist (r=−0.545; CI=−0.721 mm to −0.275 mm; P<0.001).

FIG. 3, Table 1, is a table which illustrates criteria used to define tear dysfunction subsets and normal controls.

FIG. 4, Table 2, is a table which illustrates demographic characteristics in patients with tear dysfunction and normal controls.

FIG. 5, Table 3, is a table which provides a summary of mean values of clinical ocular surface parameters, corneal sensitivity, and blink rate in patients with tear dysfunction and normal controls.

FIG. 6, Table 4, is a table which demonstrates correlations between Cochet-Bonnet corneal sensitivity, clinical parameters, and blink rate in patients with tear dysfunction.

FIG. 7, Table 5, is a table which demonstrates correlations between blink rate clinical parameters in patients with tear dysfunction.

### DETAILED DESCRIPTION

Tear instability and epithelial disease can disrupt corneal epithelial barrier function, which can affect corneal sensitivity and nerve morphology. Studies measuring corneal sensitivity in dry eye by contact and non-contact methods have reported conflicting results with either increased, decreased or no change in sensitivity. However, none of these previously reported studies stratified dry eye subjects by cause of tear dysfunction. Because corneal epithelial disease is more severe in aqueous tear deficiency than in meibomian gland disease and conjunctivochalasis, we hypothesized there may be differences in corneal sensitivity and blink rate between these subsets of tear dysfunction that may be related to severity of ocular surface epithelial disease. Corneal sensitivity and blink rate have not been compared between these distinct subsets of tear dysfunction. Evaluating corneal sensitivities amongst different subsets of tear dysfunction may prove to be important for stratifying patients for clinical trials, for determining the cause for ocular irritation/pain symptoms and perhaps for making treatment recommendations. Furthermore, the relationship between sensitivities and blink rate may provide insight into the mechanisms for increased blinking in dry eye. Testing corneal sensitivity in defined subsets of tear dysfunction may help to explain the conflicting results of previous studies that have reported both corneal hyposensitivity and hypersensitivity findings.

A non-contact esthesiometer may allow improved measurement, characterization, and treatment of patient symptoms. The objective of this study was to compare corneal sensitivity using conventional contact methods and a non-contact method implementing one embodiment of a non-contact esthesiometer in three common subtypes of tear dysfunction (aqueous tear deficiency, meibomian gland disease and conjunctivochalasis) to demonstrate the improved capability of the non-contact esthesiometer. The relationship between corneal sensitivity and irritation symptoms, blink rate, and clinical parameters was also assessed.

### Study Design

Subjects underwent a standardized tear and ocular surface evaluation in the following order that included anterior segment optical coherence tomography (OCT) as a measure of tear production and volume, respectively, fluorescein tear break-up time (TBUT) as a measure of tear stability, and corneal fluorescein and conjunctival lissamine green dye staining as measures of ocular surface epithelial cell health. Corneal and conjunctival dye staining with fluorescein and lissamine green, respectively, were performed and graded as previously reported.¹⁹ Severity of eye irritation symptoms was measured using validated questionnaires, including the ocular surface disease index (OSDI) and a 5 question visual analog scale (VAS). After standard clinical tests were performed, corneal sensitivity was measured by both Cochet-Bonnet and air jet esthesiometers, and blink rate was measured using electromyography (EMG) with signals detected by the NeuroSky™ MindBand Bluetooth device (NeuroSky, Silicon Valley, Calif.). Data from only one eye (with the worst corneal fluorescein staining) for each subject, and the right eye for normal control subjects was included in the data analysis.

### Subjects

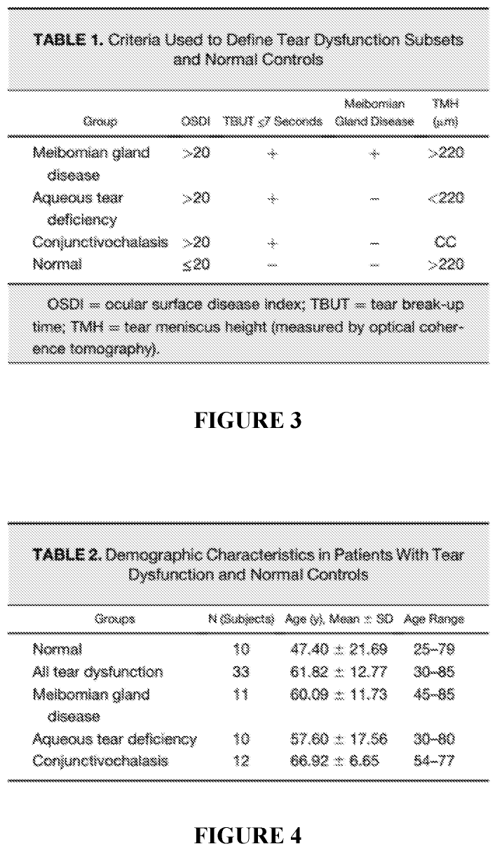

Thirty-three subjects with tear dysfunction were classified into the following groups: aqueous tear deficiency, meibomian gland disease and conjunctivochalasis according to criteria listed in FIG. 3, Table 1. The classifications were based on an ocular surface disease index (OSDI) score>20, tear break-up time (TBUT)<7 seconds, tear meniscus height measured by optical coherence tomography (OCT), and the presence (or absence) of meibomian gland disease and conjunctivochalasis.

Normal control subjects had an OSDI score≤20, no history of contact lens or eye drop use, or prior ocular surgery. They also had a TBUT≥8 seconds, and absence of fluorescein and lissamine green staining, meibomian gland disease and conjunctivochalasis on biomicroscopic examination.

Subjects were excluded if they had prior LASIK or corneal transplantation surgery, cataract surgery in the past year, punctal occlusion with plugs or cautery, a history of contact lens wear, use of topical medications other than preservative-free artificial tears, or chronic use of systemic medications known to reduce tear production. In addition, subjects were excluded if they had active ocular surface or corneal inflammation, infection, or eyelid disorders causing exposure of the ocular surface. Seventy-one patients were excluded due to these criteria.

### Optical Coherence Tomography

OCT measurement of the height of the lower tear meniscus was performed as described previously. All subjects underwent cross-sectional imaging of the lower tear meniscus prior to the instillation of drops or measurement of clinical parameters.

### Fluorescein Tear Break Up Time and Corneal Fluorescein Staining

TBUT was measured by instilling fluorescein into the lower fornix with a fluorescein strip (BioGlo, HUB, Rancho Cucamonga, Calif.) wet with preservative-free saline (Unisol; Alcon, Fort Worth, Tex.). The patient was allowed to blink at a spontaneous rate, and the elapsed time from the last blink to the appearance of the first break in the continuous layer of fluorescein, as observed under cobalt blue light through a yellow filter, was measured in seconds. Three separate measurements were taken as previously described. Corneal fluorescein staining was graded 0 to 6 in each of 5 zones (inferior, nasal, temporal, central and superior) 1 minute after fluorescein instillation, as previously reported.

### Conjunctival Lissamine Staining

The ocular surface was examined under white light illumination 1 minute after touching the inferior tarsal conjunctiva with a lissamine green strip (Green Glo 1.5 mg Lissamine green (HUB Pharmaceuticals LL Rarncho Cucamonga, Calif.) wet with preservative-free saline. Staining was graded on a scale of 0 to 3 in the exposed nasal and temporal bulbar conjunctiva with a total maximum score of 6 as previously reported.

### Corneal Sensitivity

Corneal sensitivity was measured by both Cochet-Bonnet and by air esthesiometer. A Cochet-Bonnet esthesiometer with a 0.12 mm nylon monofilament touched the center of the corneal surface at a perpendicular angle under illumination. Both eyes of each patient were tested. Patients were asked to indicate when they perceived touch. The longest length of 6.0 cm was utilized first, which corresponds to greater sensitivity. The thread length was decreased by 1.0 cm increments and the measurement repeated until sensation was felt, and it was then increased by 0.5 mm to obtain a final reading to the closest 0.5 mm.

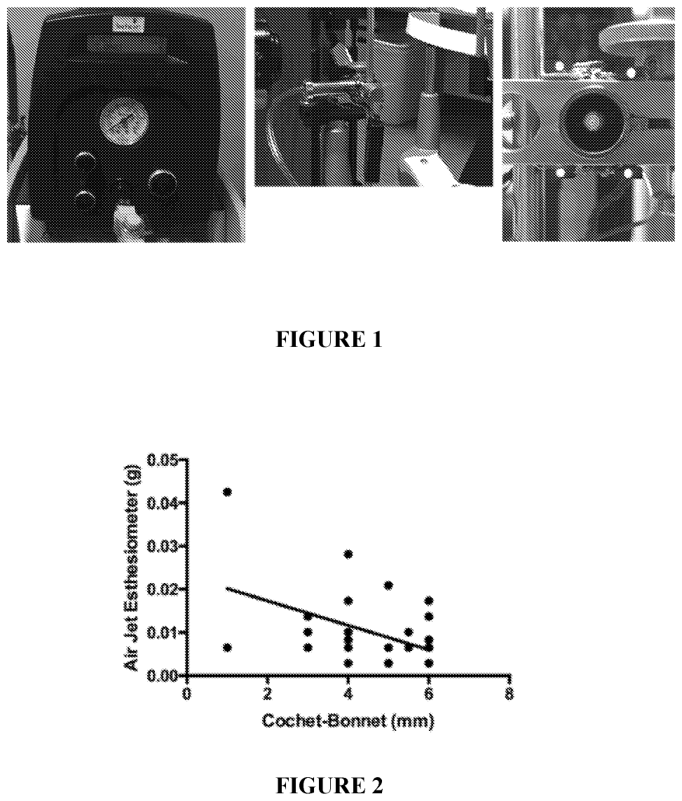

An air esthesiometer was used to evaluate corneal sensation with a non-contact method. One embodiment of an air non-contact esthesiometer is shown in FIG. 1. The air esthesiometer may include a cylinder of medical grade compressed air that is connected to an industrial pump that outputs an air stimulus at a given pressure over two seconds when its foot pedal is depressed (left). The air then travels to a hose containing a flow meter that is connected to a pipette tip that is secured on a calibrated movable mount that is attached to a stand directly mounted to a slit lamp (middle). The mount housing has 4 red light-emitting diodes centered around the pipette tip to aid in aligning the outflow stream with the center of the subject's cornea (right).

One embodiment of an esthesiometer may include a cylinder of medical grade compressed air that is connected via a unidirectional pressure regulator adjusted to 3 psi and inline filter to the OK International DX-255 Basic Digital Fluid Dispenser (OK International, Garden Grove, Calif.), which outputs the air stimulus at a given pressure over a period of two seconds when its foot pedal is depressed. The air then travels to a hose line in which the final flow of gas is adjusted with a flow meter and supplied to a 200 μL pipette tip with an internal diameter of 0.457 mm, that is attached to the end of the hose and secured on a calibrated movable mount that is attached to a stand that can be directly mounted on a Haag-Streit slit lamp (Köniz, Switzerland). The mount housing has 4 red LEDs centered around the pipette tip to aid in aligning the outflow stream with the center of the subject's cornea. During stimulation, the air stimulus was triggered by a foot pedal pressed by the investigator. The average temperature of the air released by the tip was 28° C.

To measure corneal sensitivity, subjects were seated in front of the esthesiometer tip that was positioned 5 mm away from the center of the cornea using a knob on the movable mount. The air-stimulus was applied by tapping the foot pedal that triggered an audible click by the air valve, indicating the onset of the 2-second pulse stimulus. Subjects were informed the air stimulus might be perceived as a “breeze-like” sensation beforehand. The force of the air stimulus was controlled by a knob turned in 45 degree increments and was turned each time the stimulus was not detected. Subjects were asked to report the presence or absence of sensation and to describe the sensation immediately after hearing the audible click. Subjects were instructed to blink between clicks, and the lowest detectable stimulus that elicited a response was recorded as the mechanical threshold. When a response was detected, the experimenter dialed back the knob by 45 degrees to lower the stimulus intensity and confirm the number of turns necessary to elicit the threshold stimulus. The force of the air stimulus was measured in mass (grams).

### Blink Rate Measurement

Blink rate was measured using electromyography (EMG) signals detected by the NeuroSky™ MindBand Bluetooth device (NeuroSky, Silicon Valley, Calif.). The MindBand was placed on the subject's forehead and the dry electrodes on the MindBand measured the changing electrical potential of the orbicularis muscles during blinks.

The threshold for detecting a blink was set prior to recording the patient's average blink rate per minute and was adjusted for each individual. Subjects were asked to look straight ahead, in a relaxed manner, without any additional activity for 5 minutes. Patients were asked to avoid speaking, moving extremities, or making facial expressions. Excessive movements during the measurement period were excluded from the data analysis, and only blink rates from minutes 2-4 were used for calculations. Blinks were measured and recorded as blinks/minute. The blink count readings were verified by manual blink counting for each patient.

Testing was performed in the following order: measurement of tear meniscus height by OCT, blink rate measurement, corneal sensitivity by air esthesiometer, tear break-up time, corneal fluorescein and conjunctival lissamine green staining, and, corneal sensitivity by Cochet-Bonnet.

### Data Analysis

The data was analyzed using GraphPad (Prism 6.0, La Jolla, Calif.). Normality distribution of data sets was determined using the D'Agostino-Pearson normality omnibus test. Many, but not all of our parameters were normally distributed, thus both parametric (Pearson's correlation coefficient and ANOVA), and non-parametric tests (Spearman's rank correlation coefficient, Mann-Whitney, and the Kruskal-Wallis test) were performed. Because the results of parametric and non-parametric tests were similar, the mean values of corneal sensitivity, blink rate, and clinical parameters were compared between tear dysfunction subtypes and control group using ANOVA. All data sets included measurements from interval scales, so the Pearson correlation coefficient (R) was calculated to assess the relationship between corneal sensitivity and irritation symptoms, blink rate, and clinical parameters within the entire tear dysfunction group and within each subtype. A P value of ≤0.05 was considered to be statistically significant.

### Results of Study Population

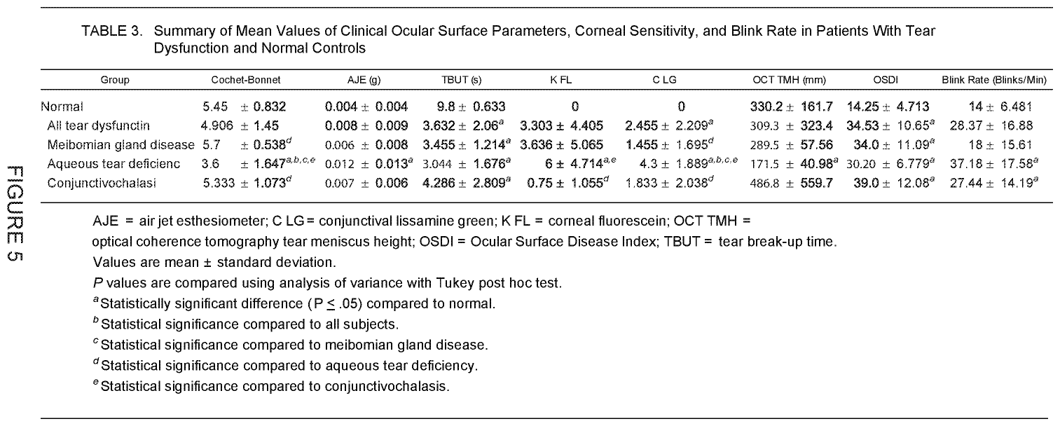

The demographic features for control and tear dysfunction subjects are presented in FIG. 4, Table 2. Age ranged from 30 to 85 years (61.82±12.77 [mean±SD]) in the 33 tear dysfunction subjects, and 25 to 79 years (47.4±21.69 [mean±SD]) in the 10 control subjects. There was a statistically significant difference in age between all tear dysfunction (61.82 years) and control (47.4 years) subjects (P=0.006), and between conjunctivochalasis (66.92 years) and controls (47.4 years) (P=0.004). There was no difference in age between either meibomian gland disease or aqueous tear deficiency and the control group and there was no statistically significant difference in mean age between the tear dysfunction groups.

Results of Mean Value Comparisons for Corneal Sensitivity

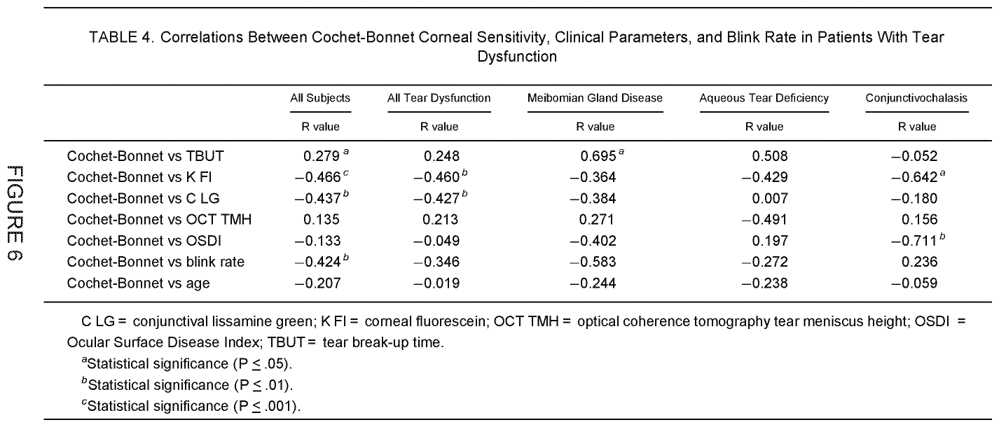

For each group, the mean and standard deviation values for corneal sensitivity measured with both methods, clinical parameters of tear function, ocular surface disease and blink rate are shown in FIG. 5, Table 3. When compared with the mean corneal sensitivity threshold in the control group using the Cochet-Bonnet (5.450 mm; 95% confidence interval (CI)=4.86 mm to 6.04 mm), there was a significantly higher threshold in the aqueous tear deficiency group (3.6mm; CI=2.42 mm to 4.78 mm; P<0.003). When compared with the mean threshold in the control subjects using the air esthesiometer (3.62 mg), there was also a significantly higher threshold in the aqueous tear deficiency group (11.7 mg; CI=2.18 mg to 21.2 mg; P=0.046).

### Results Showing Correlation Between Cochet-Bonnet and Air Let Esthesiometers

FIG. 2 shows a significant correlation between our prototype air esthesiometer and the Cochet-Bonnet was found for dry eye subjects (r=−0.512; CI=−0.730 mm to −0.199 mm; P<0.001). In addition, there was significant correlation between our air esthesiometer and the Cochet-Bonnet for all subjects (r=−0.545; CI=−0.721 mm to −0.275 mm; P<0.001).

### Results of Mean Values Comparison for Blink Rate

When compared with mean blink rate in the control group (14 blinks/min; CI=9.02 blinks/min−19.0 blinks/min), significantly higher mean blink rates were measured in both the aqueous tear deficiency group (37.18 blinks/min; CI=22.5 blinks/min to 51.9 blinks/min; P=0.001) and conjunctivochalasis group (27.44 blinks/min; CI=16.5 blinks/min to 38.3 blinks/min; P=0.01). There was no significant difference in blink rate between meibomian gland disease (18 blinks/min; CI=1.52 blinks/min to 34.4 blinks/min; P=0.250) and control.

### Results of Correlations Between Corneal Sensitivity, Blink Rate, and Clinical Parameters

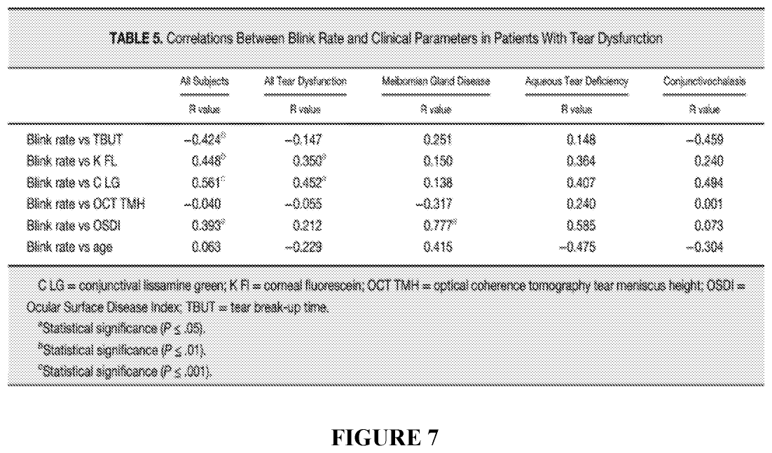

The correlations between corneal sensitivity, blink rate, and clinical parameters are presented in FIGS. 6 and 7, Tables 4 and 5. Reduced corneal sensitivity with the Cochet-Bonnet esthesiometer was significantly correlated with more rapid TBUT, ocular surface dye staining and blink rate, while reduced sensitivity with the air esthesiometer correlated with more rapid TBUT, irritation symptoms measured by the OSDI and blink rate. In addition, there was a significant correlation between the air jet esthesiometer and TBUT, OSDI, and blink rate in all subjects. Moreover, there was a significant positive correlation (P=0.043) between the air jet esthesiometer and OSDI in the mebomian gland disease subset.

Mean value comparisons of corneal sensitivity measured with both methods, clinical parameters of tear function and blink rate between each subtype of tear dysfunction are shown in FIG. 5, Table 3. When comparing mean corneal sensitivity threshold using the Cochet-Bonnet, there was a significantly higher threshold in the aqueous tear deficiency group compared to the conjuctivochalasis group (p=0.004) or the meibomian gland disease group (p<0.001). The aqueous tear deficiency group had significantly higher corneal staining than the conjuctivochalasis group (p=0.006). The aqueous tear deficiency group also had significantly higher conjuctival lissamine green staining compared to either the meibomian gland disease group (p=0.002) or conjuctivochalasis group (p=0.007). There were no significant differences between each subtype of tear dysfunction groups for TBUT, OSDI, and blink rate.

Results of Correlation of blink rate with clinical parameters

In all subjects, blink rate positively correlated with corneal staining score (R=+0.448; CI=0.177 to 0.689; P=0.005), conjunctival staining score (R=+0.561; CI=0.263 to 0.761; P<0.001), and irritation score measured with the OSDI questionnaire (R=+0.393; CI=0.031 to 0.664; P=0.018), and inversely correlated with TBUT (R=−0.424; CI=−0.673 to −0.086; P=0.008) as shown in FIG. 7, Table 5.

### Discussion of Results and Non-Contact Esthesiometer

In this study, we found corneal sensitivity to be reduced in the aqueous tear deficiency subset. Reduced corneal sensitivity was associated with greater eye irritation symptoms, tear instability, ocular surface disease, and blink rate. Previously published studies that evaluated corneal sensitivity in patients with dry eye have reported conflicting results. Eleven studies have shown subjects with dry eye symptoms to have hypoesthesia; however, three other studies have reported the opposite. Additionally, Chen and Simpson reported no difference in corneal sensitivity in soft contact lens wearers with and without symptoms of dry eye and Tuisku and associates found no difference in corneal sensitivity between LASIK patients who complained of dye eye symptoms and normal controls. Several studies regarding corneal sensitivity in association with changes in the subbasal nerve plexus reported corneal hyposensitivity, and one described improvement in sensitivity following cyclosporine therapy. In our study, the aqueous tear deficiency group demonstrated corneal hyposensitivity with both the Cochet-Bonnet contact esthesiometer and the non-contact air esthesiometer. In contrast, the meibomian gland disease and conjunctivochalasis groups had corneal sensitivity thresholds similar to control subjects. The aqueous tear deficiency group had a lower tear meniscus height and higher corneal and conjunctival staining than the meibomian gland disease and conjunctivochalasis groups, which may contribute to the differences in corneal sensitivity observed between the different subtypes of tear dysfunction. The decreased corneal sensitivity found in the aqueous tear deficiency group was associated with increased corneal and conjunctival dye staining, which is consistent with other studies. It appears that many of the previously published studies that evaluated corneal sensitivity in dry eyes did not use stringent criteria to distinguish between different subtypes of tear dysfunction, classifying subjects as dry eye, LASIK, Sjögren syndrome (SS), rheumatoid arthritis and rarely aqueous tear deficiency. Our finding of decreased corneal sensitivity only in the aqueous tear deficiency group that was defined by a low OCT-measured tear volume may be one possible explanation for the conflicting corneal sensitivity findings previously reported. Certain studies, particularly those evaluating patients with SS, most likely evaluated primarily aqueous tear deficiency patients, while others may have had included subjects with meibomian gland disease and conjunctivochalasis. Specifically, because only a few studies distinguished between SS and non-SS patients, we can only be certain that those particular studies evaluating SS consisted of an aqueous tear deficiency population. From the fourteen studies that have reported corneal sensitivity findings in dry eye disease, only the studies by Benitez-Del-Castillo and associates and by Toker and Asfuroglu enrolled approximately 50% or more SS patients who were found to have corneal hyposensitivity. These findings are consistent with our study and support the hypothesis that greater and more chronic corneal epithelial disease may lead to degeneration of corneal nerve endings and reduced corneal sensitivity. Indeed, reduced density of the subbasal nerve plexus has been found in aqueous tear deficiency with and without SS.

Another possibility is that chronic inflammation induced by tear dysfunction and epithelial disease may contribute to corneal nerve degeneration and reduced sensitivity. It remains to be determined if corneal sensitivity is normal or even increased in subjects with marked corneal epithelial disease from recent onset aqueous tear deficiency before the nerve endings degenerate.

The contradictory reports could also be due to differences in methods used to measure corneal sensitivity and in criteria used to define dry eye patients. Because the Belmonte air esthesiometer is not commercially available, we designed our own air esthesiometer. Although our prototype air esthesiometer has certain differences from the Belmonte gas esthesiometer, both esthesiometers deliver the same type of controlled air jet stimulus. Differences between the instruments include the internal diameter of the air outlet that is 0.457 mm in our model and reported to be 0.8 mm in the Belmonte instrument and the ability to change temperature of the air stimulus in the Belmonte instrument. Our instrument also had LED lights around the outlet that assisted in delivering the stimulus to the center of the cornea. We found a significant correlation between our prototype air esthesiometer and the Cochet-Bonnet esthesiometer in subjects with tear dysfunction and in all subjects as shown in FIG. 2. This finding suggests that use of contact or non-contact esthesiometers may not be the cause for the conflicting results of previously reported studies evaluating corneal sensitivity in dry eye. The use of both contact and non-contact methods to measure corneal sensitivity is a unique feature of our study.

Our use of OCT tear meniscus height as an indirect measure of tear volume enabled us to accurately measure the amount of tears in the inferior tear meniscus and is another unique feature of our study. This allowed for better classification of the tear dysfunction groups into aqueous sufficient or aqueous deficient. Together with the clinical examination, it also identified conjunctivochalasis. The previously repeated studies did not measure tear meniscus height by OCT. Our findings suggest that using OCT to identify patients with aqueous tear deficiency may identify those at risk for developing corneal hypoesthesia.

Interestingly, our study showed that decreased corneal sensitivity was associated with increased ocular surface irritation symptoms with the air esthesiometer, but not with the Cochet-Bonnet. Although both the Cochet-Bonnet and non-invasive air esthesiometer stimulate mechanoreceptors, the air esthesiometer may stimulate other receptors, such as polymodal and cold thermoreceptors whose hyposensitivity may be responsible for the inverse correlation between corneal sensitivity and irritation symptoms that was only seen with the air esthesiometer. In contrast, a previously reported study that used a non-contact air esthesiometer noted increased corneal sensitivity that correlated with increased ocular surface symptoms. Our results seem counterintuitive, as we would expect patients with reduced corneal sensitivity to report less severe eye irritation symptoms than those with normal or increased sensitivities. The basis for our findings remains to be determined. As suggested by previous studies, hyposensitivity and hypersensitivity may be indicators of different stages of dry eye disease, which may help explain the paradoxical finding. Cold thermoreceptors in the cornea have been found to stimulate basal tear secretion in mice and their stimulation from the normal temperature oscillations during interblink intervals in healthy eyes under normal environmental conditions has been hypothesized to give a sensation of ocular comfort or wetness. Reduced sensitivity of these nociceptors to the stimulus delivered by our air esthesiometer that is cooler than the normal cornea temperature of 34° C. could lessen the physiological stimulation of tear secretion and possibly contribute to the increased discomfort reported by these subjects.

Similar to previously reported studies, blink rate was found to be increased in aqueous deficient dry eye and we also noted it to be increased in conjunctivochalasis, where central inferior tear meniscus height has previously been found to be normal. Another interesting finding was that reduced corneal sensitivity by both contact and non-contact methods was associated with more frequent blinking. Increased blink rate was positively correlated with severity of irritation symptoms, corneal fluorescein staining and inversely with TBUT.

Our finding that decreased corneal sensitivity correlated with increased blink rate in tear dysfunction is surprising. A study by Toda and associates measured corneal sensitivities and blink rates in 64 patients following LASIK surgery. They discovered that the majority of their patients displayed hyposensitivity at 1 and 3 months with a return to baseline sensitivities by 6 months; however, blink rate in these patients was significantly decreased from 3 months onward. In addition, Collins and associates studied the relationship between corneal sensitivity and blink rate in 9 patients by measuring blink rate both before and after use of a topical corneal anesthetic. They found a significant decrease in blink rate after the anesthetic was applied. Based on these findings, we would have expected a decreased blink rate in the aqueous tear deficiency group. However; it is possible that increased blink rate is triggered by factors other than corneal sensation. One potential trigger for increased blink rate is rapid TBUT. In addition to triggering nerve stimulation in areas of tear disruption, tear break up may also increase light scattering and cause patients with tear dysfunction to blink more frequently to improve their quality of vision. Al-Abdulmunem and others suggested that there might be both cortical control and ocular surface control mechanisms driving blinking with the latter predominating in dry eye patients.

In conclusion, our study demonstrates the importance of classifying tear dysfunction groups into subsets, which is often neglected in studies correlating clinical parameters in tear dysfunction. Our study demonstrates that there are significant differences in corneal sensitivity and blink rate between meibomian gland disease, aqueous tear deficiency, and conjunctivochalasis. Therefore, it does not seem appropriate to group these disorders into a generic dry eye category when studying these parameters. Although our findings of decreased corneal sensitivity with increased symptoms and increased blink rate is a surprising discovery, our study is the first to not only distinguish between tear dysfunction subcategories in order to eliminate confounding disease processes, but also to set stringent criteria in regards to measuring corneal sensitivity with both contact and non-contact methods in both eyes, evaluate tear meniscus height by OCT, and incorporate blink rate into our study design. Future studies using larger sample sizes and our tear dysfunction classifications may determine the effects of treatment. These studies may establish how corneal sensitivity changes over time amongst these subsets, and how these changes correspond to blink rate. Our findings have helped set the framework for further research into the causes for eye irritation and increased blink rate in tear dysfunction conditions.

The methods described above includes steps in particular orders. However, other steps and methods may be conceived that are equivalent in function, logic, or effect to one or more steps, or portions thereof, of the illustrated method. Additionally, these descriptions should be understood not to limit the scope of the method. For instance, methods may include a waiting or monitoring period of unspecified duration between enumerated steps of the method. Additionally, the order in which steps of a particular method occurs may or may not strictly adhere to the order described herein.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. An apparatus, comprising:
a compressed air source;
a line coupled to the compressed air source and configured to couple to an outlet tip, wherein the line is configured to supply compressed air from the compressed air source through the line to exit through the outlet tip.

2. The apparatus of claim 1, further comprising a valve coupled between the line and the outlet tip and configured to adjust an airflow rate of the compressed air exiting through the outlet tip.

3. The apparatus of claim 2, wherein the valve is configured to provide an airflow rate of approximately 3 psi at the outlet tip.

4. The apparatus of claim 1, further comprising an inline filter coupled between the compressed air source and the line.

5. The apparatus of claim 1, wherein the compressed air source comprises at least one of a compressed air tank and an air compressor.

6. The apparatus of claim 1, further comprising a stand configured to secure the outlet tip and configured to provide horizontal and vertical movement of the outlet tip.

7. The apparatus of claim 6, wherein the metal stand is configured to attach to a slit lamp and comprises four light emitting diode (LED)-based bulbs.

8. The apparatus of claim 1, wherein the outlet tip comprises a disposable 200-microliter-filter pipette tip.

9. The apparatus of claim 1, further comprising a user input device, wherein activation of the user input device triggers a 2-second stream of air to exit the outlet tip.

10. The apparatus of claim 9, wherein the user input device comprises a foot pedal.

11. A method of operating an air esthesiometer, comprising:
placing a patient's head into a stand;
adjusting a slit lamp horizontally and vertically to align an outlet tip with a center of the patient's eye;
advancing the outlet tip towards the patient's eye to a desired distance from the patient's eye;
triggering an outlet of compressed air through the outlet tip towards the patients' eye; and
recording the patient's response to the compressed air.

12. The method of claim 11, further comprising increasing a pressure of the air from the outlet tip; and repeating the steps of triggering the outlet of compressed air and recording the patient's response to the compressed air.

13. The method of claim 11, wherein the step of adjusting the stand/slit lamp to align the outlet tip with the center of the patient's eye comprises using the reflection of LED bulbs in the patient's cornea.

14. The method of claim 11, wherein the step of triggering an outlet of compressed air comprises outputting two seconds of compressed air at approximately 3 psi towards the patient's eye.
